(19) United States
(12) Patent Application Publication
(10) Pub. No.: US 2025/0076307 A1
(43) Pub. Date: Mar. 6, 2025

(54) METHODS FOR CHARACTERIZING A PROTEIN OF INTEREST

(71) Applicant: REGENERON PHARMACEUTICALS, INC., Tarrytown, NY (US)

(72) Inventors: KLEINBERG Andrew, Roslyn, NY (US); MAO Yuan, Hartsdale, NY (US); LI Ning, New Canaan, CT (US)

(21) Appl. No.: 18/817,850

(22) Filed: Aug. 28, 2024

Related U.S. Application Data
(60) Provisional application No. 63/535,227, filed on Aug. 29, 2023.

(51) Int. Cl. G01N 33/68; C12Q 1/37

(52) U.S. Cl. CPC G01N 33/6848; C12Q 1/37; G01N 33/6854; G01N 2333/976

(57) ABSTRACT

Methods can be conducted to prevent disulfide scrambling in liquid chromatography-mass spectrometry analysis of a protein of interest. In particular, methods can be conducted by employing a NEM analog, such as maleimide, and a heavy isotope counterpart of the NEM analog, such as a heavy isotope substituted maleimide, to alkylate a protein of interest and to alkylate a peptide digest of the protein of interest under conditions that prevent disulfide scrambling and subjecting the samples to a liquid chromatography-mass spectrometry analysis.

### CROSS-REFERENCE TO RELATED APPLICATIONS

This application claims the benefit of and priority to U.S. Provisional Application No. 63/535,227, filed on Aug. 29, 2023, the contents of which are incorporated by reference in its entirety herein.

### SEQUENCE LISTING

The instant application contains a Sequence Listing which has been submitted electronically in XML file format and is hereby incorporated by reference in its entirety. Said XML copy, created on Aug. 8, 2024, is named 086939_500802_SL.xml and is 7,619 bytes in size.

### FIELD

This application relates to methods for carrying out protein characterization, including site-specific free thiol quantitation.

### BACKGROUND

Mass spectrometry (MS) has become an increasingly important technique to analyze proteins. In popular bottom-up MS-based proteomics, reduction and alkylation are routine steps to facilitate peptide identification. However, side reactions may occur, which compromise the experimental results.

Sample preparation is a critical step in bottom-up MS-based proteomics. One challenge in the use of MS-based proteomics is prevention of disulfide bond scrambling during a sample preparation. Disulfide scrambling causes the rearrangement of pre-existing free thiols into different ratios. Paired cysteines may no longer show matching free thiol levels. Numerous methods have been developed, in the past, to address this challenge. It has been noted that temperature, pH, and the availability of free cysteines are factors that can be controlled during sample preparation to prevent the formation of non-native disulfide bonds.

It is widely accepted that pH affects disulfide bond or cysteine reactivity, even at room temperature, and should be carefully controlled during sample preparation. The reported methods teach sample preparation at slightly acidic pH, stating that at alkaline pH, free thiols are deprotonated, and the resulting thiolate anions are oxidized or react with adjacent disulfide bonds (thiol/disulfide exchange) to form new, non-native disulfide bonds. After the low pH protein alkylation, the pH can be increased for protein digestion, which adds an extra step, or the low pH can be maintained for the digestion conditions.

While protein digestion conducted entirely under acidic conditions can minimize disulfide scrambling, such acid conditions create digestion peptide profiles with significant differences (e.g., non-specific cleavages) compared to traditional alkaline-pH digests. Thus, there is a continuing need in the art for efficient methods for protein preparation, without requiring a low pH protein alkylation.

### SUMMARY

Advantageously, a method has been developed for characterizing a protein of interest, while minimizing or preventing the formation of sample preparation-induced disulfide scrambling. The method includes differentially alkylating a sample with a N-ethylmaleimide (NEM) analog and its heavy isotope substituted counterpart. For example, methods of the present disclosure include alkylating a protein of interest in a first sample with an initial alkylating agent to form a second sample with an alkylated protein of interest. The initial alkylating agent is either: (i) a NEM analog, or (ii) a heavy isotope substituted form of the NEM analog. Such a heavy isotope substituted form of the NEM analog can include a NEM analog substituted with a heavy isotope of carbon, e.g., a carbon-13 and/or carbon-14 substituted NEM analog, for example.

The method can continue by digesting the alkylated protein of interest with at least one digestive enzyme to form a peptide digest; and alkylating the peptide digest with a differential alkylating agent to form a third sample. The differential alkylating agent is either: (i) the NEM analog, or (ii) the heavy isotope substituted form of the NEM analog that was not used as the initial alkylating agent. That is, if the initial alkylating agent is (i) the NEM analog, the differential alkylating agent is (ii) the heavy isotope substituted form of the NEM analog. Conversely, if the initial alkylating agent is (ii) the heavy isotope substituted form of the NEM analog, the differential alkylating agent is (i) the NEM analog.

The method can continue by subjecting the third sample to analysis using liquid chromatography-mass spectrometry, e.g., subjecting the sample to a liquid chromatography system coupled to a mass spectrometer and performing an analysis of the third sample. Advantageously, the methods can include site-specific free thiol quantitation, or characterizing a protein of interest, or a non-reduced peptide mapping of the protein of interest.

In an implementation, a method for site-specific free thiol quantitation of a protein of interest in a first sample can include: alkylating the protein of interest in the first sample with an alkylating agent to form a second sample with an alkylated protein of interest, wherein the alkylating agent is either: (i) a NEM analog, or (ii) a heavy isotope substituted form of the NEM analog; digesting the alkylated protein of interest with at least one digestive enzyme to form a peptide digest; alkylating the peptide digest with a differential alkylating agent to form a third sample, wherein the differential alkylating agent is either (i) the NEM analog, or (ii) the heavy isotope substituted form of the NEM analog; and subjecting the third sample to analysis using liquid chromatography-mass spectrometry to quantify a site-specific free thiol in the first sample. Advantageously, the method for site-specific free thiol quantitation of the protein of interest in the sample can determine native free thiol levels.

In another implementation, a method for characterizing a protein of interest in a sample can include: alkylating the protein of interest in the first sample with an alkylating agent to form a second sample with an alkylated protein of interest, wherein the alkylating agent is either: (i) a NEM analog, or (ii) a heavy isotope substituted form of the NEM analog; digesting the alkylated protein of interest with at least one digestive enzyme to form a peptide digest; alkylating the peptide digest with a differential alkylating agent to form a third sample, wherein the differential alkylating agent is either: (i) the NEM analog, or (ii) the heavy isotope substituted form of the NEM analog; and subjecting the third sample to analysis using liquid chromatography-mass spectrometry to characterize the protein of interest in the first sample. Characterizing the protein of interest can include subjecting the third sample to analysis using liquid chromatography-mass spectrometry to obtain a non-reduced peptide mapping of the protein of interest. Advantageously, the method can perform the peptide mapping of the protein of interest in the sample while determining native free thiol levels. The method can be performed as a non-reduced peptide mapping of the protein of interest.

In a still further implementation, a method can include forming an alkylated product by either: alkylating the protein of interest with a carbon 13 substituted NEM analog to form a ¹³C-NEM analog alkylated protein of interest, or alkylating a peptide digest of the protein of interest with a carbon 13 substituted NEM analog to form a ¹³C-NEM analog alkylated peptide digest. Such a carbon 13 substituted NEM analog can include ¹³C₄-maleimide. The method can characterize the protein of interest in a first sample such as by subjecting the alkylated product to analysis using liquid chromatography-mass spectrometry to characterize the protein of interest.

Additional aspects of the present disclosure include a 13C-NEM analog alkylated product. Such a product can include a ¹³C-NEM analog alkylated protein of interest, such as a ¹³C₄-maleimide alkylated protein of interest, a ¹³C-NEM analog alkylated peptide digest, such as a ¹³C-NEM analog alkylated peptide digest, or any combination thereof.

In accordance with the present disclosure, one or more of the following features, individually or combined, can be included in any of the methods. For example, a buffer exchange can be performed after alkylating the protein of interest in the first sample, e.g., to remove remaining alkylating agent after alkylating the protein of interest. Further, in some aspects, the peptide digest can be reduced with a reducing agent, e.g., reducing the peptide digest can be carried out with the reducing agent of tris(2-carboxyethyl)phosphine. The peptide digest can be reduced prior to alkylating the peptide digest with the differential alkylating agent. In other aspects, the heavy isotope substituted form of the NEM analog can include a NEM analog substituted with a heavy isotope of carbon, e.g., a carbon-13 and/or carbon-14 substituted NEM analog, for example.

In one aspect, the NEM analog is less hydrophobic than NEM. In another aspect, the NEM analog has a retention time less than the retention time of NEM. In yet another aspect, the NEM analog is maleimide and the heavy isotope substituted form of maleimide comprises fully carbon-13 substituted maleimide, i.e., ¹³C₄-maleimide.

In one aspect, the alkylating agent used to alkylate the protein of interest is the heavy isotope substituted form of the NEM analog and the differential alkylating agent used to alkylate the protein digest is the NEM analog. In another aspect, the alkylating agent used to alkylate the protein of interest is the NEM analog and the differential alkylating agent used to alkylate the protein digest is the heavy isotope substituted form of the NEM analog.

In some aspects, alkylating the protein of interest or alkylating the peptide digest independently can be conducted at a temperature and time sufficient to alkylate the protein of interest, or alkylate the peptide digest, respectively. For example, the alkylating reaction with either: (i) the NEM analog, or (ii) the heavy isotope substituted form of the NEM analog can be conducted from about room temperature (about 20° C.) to about 80° C., e.g., from about 30° C. to about 70° C., such as at about 50° C. The alkylating reaction with either (i) the NEM analog, or (ii) the heavy isotope substituted form of the NEM analog can be conducted for at least about 5 minutes such as for at least about 20 minutes, 30 minutes, etc. In addition, or separately, alkylating the protein of interest or alkylating the peptide digest can be conducted in a medium having an acidic pH, i.e., a pH less than 7 such as between a pH of about 3 to a pH of about 6.5.

In another aspect, alkylating the protein of interest or alkylating the peptide digest can be conducted in which the alkylating agent (either (i) the NEM analog, or (ii) the heavy isotope substituted form of the NEM analog) is at a concentration of about 1 mM to about 500 mM, e.g., from about 1 mM to about 300 mM, from about 1 mM to about 100 mM, from about 1 mM to about 20 mM, and any values therein and therebetween.

In one aspect, the at least one digestive enzyme is trypsin. In another aspect, the at least one digestive enzyme includes Lys-C. In yet another aspect, the at least one digestive enzyme includes Lys-C and trypsin.

In one aspect, digestion can be conducted in a pH that can range from acidic (e.g., <pH 7) to alkaline (e.g., >pH 7), such as a pH from about 3 to about 9. For example, the digestion can be conducted at a pH between about 7 and about 8. In a particular aspect, the digestion can be conducted at a pH between about 7 and about 7.5. In another aspect, the digestion can be conducted at a pH between about 5 and about 6. In a particular aspect, the digestion can be conducted at a pH between about 5.3 and about 7.

In one aspect, subjecting the sample for analysis involves using a liquid chromatography system comprising reversed phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, said mass spectrometer is an electrospray ionization mass spectrometer, nano-electrospray ionization mass spectrometer, or an Orbitrap-based mass spectrometer, wherein said mass spectrometer is coupled to said liquid chromatography system.

In one aspect, the protein of interest is an antibody. In a particular aspect, the protein of interest is a monoclonal antibody or a bispecific antibody. In another aspect, the protein of interest is a recombinant protein.

Additional advantages of the present disclosure will become readily apparent to those skilled in the art from the following detailed description, wherein only certain embodiments are shown and described, simply by way of illustration of carrying out certain subject matter. As will be realized, the invention is capable of other and different embodiments, and its several details are capable of modifications in various respects, all without departing from the invention. Accordingly, the drawings and description are to be regarded as illustrative in nature, and not as restrictive.

### BRIEF DESCRIPTION OF THE DRAWINGS

FIG. 1 illustrates disulfide reduction with an enzyme in a typical experiment wherein reduced disulfide bonds create pairs of free thiols.

FIG. 2 shows the structures of exemplary NEM analogs that can be used in practicing certain aspects of the present disclosure.

FIGS. 3A and 3B show common free thiol alkylation mechanisms for alkylation reactions carried out using nucleophilic substitution using 2-iodoacetamide (IAM) and Michael addition using NEM. FIG. 3A illustrates a nucleophilic substitution (e.g., IAM) which is a slow mechanism compared to thio-disulfide exchange. FIG. 3B illustrates a Michael Addition (e.g., NEM). In FIG. 3B, the strained maleimide ring structure is more reactive than other Michael acceptors, the free thio alkylation occurs much faster than thiol-disulfide exchange, and the nature free thiol levels are preserved.

FIG. 4 shows a graph of alkylation of free thiol and scrambling observed for a monoclonal antibody mAb1 for IAM and NEM, according to aspects of the present disclosure. FIG. 1 discloses SEQ ID NOs: 1 and 2.

FIG. 5 shows structures of exemplary NEM analogs and heavy isotope substituted forms of the NEM analogs.

FIGS. 6A and 6B show UV chromatograms of peptide mapping analysis of mAb1 using a deuterated NEM (FIG. 6A) and a ¹³C₄-labelled maleimide (FIG. 6B), according to an exemplary embodiment. FIGS. 6A and 6B disclose SEQ ID NOs: 3-6.

FIG. 7 shows an isotope purity profile for ¹³C₄-substituted maleimide. FIG. 7 discloses SEQ ID NOs: 5 and 6.

FIG. 8 shows free thiol data for mAb2 peptide mapping, according to an exemplary embodiment.

FIG. 9 shows free thiol data for mAb1 peptide mapping, according to an exemplary embodiment.

FIG. 10 shows free thiol data for mAb3 peptide mapping, according to an exemplary embodiment.

### DETAILED DESCRIPTION

Characterization of protein product quality attributes (PQAs) is important due to the large size and complex heterogeneity of this increasingly popular class of therapeutics. One such PQA is the proper formation of classical disulfide bond structures. Deviations from the canonical IgG disulfide conformation, including non-classical disulfide bonding (scrambling), may negatively impact a protein's structure, stability, and biological efficacy (Zhang et al., 2011, Biotechnol Adv, 29 (6): 923-9; Liu et al., 2012, MAbs, 4 (1): 17-23; Liu et al., 2007, Biotechnol Lett, 29 (11): 611-22; Brych et al., 2010, J Pharm Sci, 99 (2): 764-81; Mamathambika and Bardwell, 2008, Annu Rev Cell Dev Biol, 24:211-35; Zhang et al., 2012, Anal Chem, 84 (16): 7112-23; Van Buren et al., 2009, J Pharm Sci, 98 (9): 3013-30; Zhang et al., 2019, Protein Expr Purif, 164:105459).

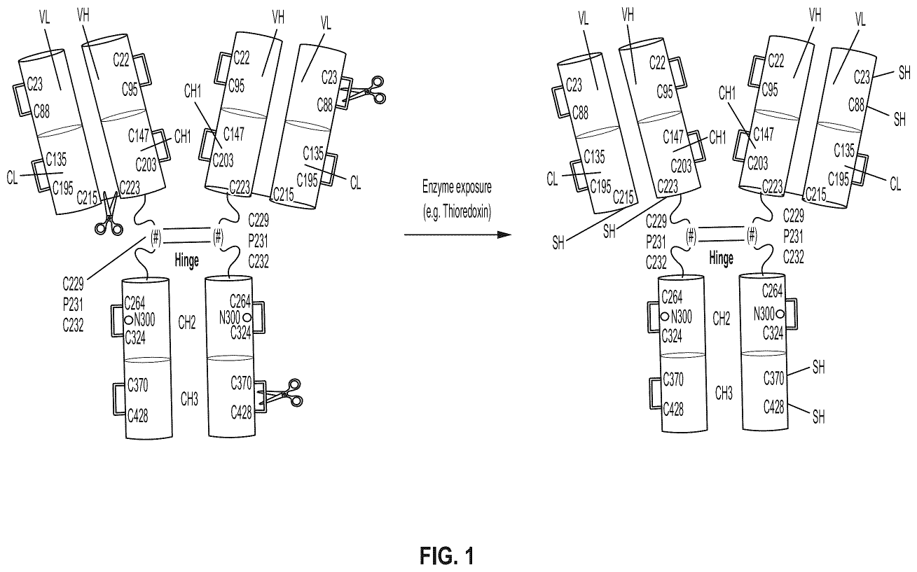

Disulfide bond conformation is highly conserved in accordance with each IgG subclass (Milstein, 1966, Biochem J, 101 (2): 338-51; Pinck and Milstein, 1967, Nature, 216 (5118): 941-2; Frangione and Milstein, 1968, J Mol Biol, 33 (3): 893-906; Frangione et al., 1969, Nature, 221 (5176): 145-8). For example, IgG1 molecules have a four-chain structure composed of two heavy chains (HCs) and two light chains (LCs) covalently linked by inter-chain disulfide bonds, as shown in FIG. 1. In addition to the inter-chain disulfide bonds, one intra-chain disulfide bond is present and is shielded within each β-barrel domain of the HC and LC polypeptides (Zhang et al., 2002, Anal Biochem, 311 (1): 1-9). In the hinge region, the two HCs are covalently linked by two inter-chain disulfide bonds.

A typical therapeutic mAb has a molecular weight of about 140 kDa, rendering traditional disulfide bond mapping methods less applicable, such as NMR (Klaus et al., 1993, J Mol Biol, 232 (3): 897-906), X-ray crystallography (Jones et al., 1997, Methods Enzymol, 277:173-208), and Edman sequencing (Haniu et al., 1994, Int J Pept Protein Res, 43 (1): 81-6). The rapid evolution of liquid chromatography-mass spectrometry (LC-MS) and its successful implementation in biomolecule analysis has enabled in-depth profiling of mAb PQAs, including canonical disulfide bond formation and identification of non-classical disulfide features like disulfide bond scrambling, free thiol, and trisulfide bond formation. The most common LC-MS approach to study mAb disulfide bonds, known as non-reduced peptide mapping, is a modified version of the conventional reduced peptide mapping approach with no disulfide reduction step and lower amount of thiol alkylating agent (Li et al., 2015, State-of-the-Art and Emerging Technologies for Therapeutic Monoclonal Antibody Characterization Volume 2. Biopharmaceutical Characterization: The NISTmAb Case Study, pp. 119-183; Formolo et al., 2015, State-of-the-Art and Emerging Technologies for Therapeutic Monoclonal Antibody Characterization Volume 2. Biopharmaceutical Characterization: The NISTmAb Case Study, pp. 1-62). Trypsin is the most commonly used digestive enzyme due to its high specificity, efficiency, and propensity to generate peptides of appropriate length for MS analysis. The resulting method enzymatically cleaves the mAb into peptide species, with any potential disulfide bonds remaining intact. All peptides are then analyzed by LC-MS, where a UV detector generates a “peptide fingerprint” by measuring the UV absorbance of the eluting analytes according to their retention times, and a mass spectrometer ionizes these analytes and records their mass-to-charge ratios (m/z). High-resolution accurate-mass (HRAM) mass spectrometers with tandem mass spectrometry (MS²) capabilities coupled to advanced protein/peptide identification algorithms like Byonic have simplified peptide mapping analysis so that even sensitive identification of disulfide-linked peptides and site-specific identification of free thiol are now routine.

The high selectivity and sensitivity of non-reduced peptide mapping inherits a disadvantage associated with reduced peptide mapping: experimental conditions and reagents can sometimes induce confounding chemical modifications into peptide sequences if the method is not thoroughly optimized and carefully developed. For non-reduced peptide mapping, scrambled disulfide artifacts were found to be associated with sample preparation steps, such as denaturation by heating and/or enzymatic digestion conditions at alkaline pH. These experimentally introduced scrambled disulfide artifacts may lead to false interpretations or conclusions regarding their pre-existing levels in the native therapeutic mAbs (Liu et al., 2007; Zhang et al., 2002, Anal Biochem, 311 (1): 1-9; Wu and Watson, 1997, Protein Sci, 6 (2): 391-8).

To reduce disulfide scrambling artifacts during non-reduced analyses, several strategies have been developed. The simplest approach is to alkylate free cysteine using an excess amount of iodoacetamide, which essentially caps all endogenous free thiols as well as artifact thiols before any scrambling can occur. FIG. 1 shows the typical formation of free thiols in an antibody. However, this simple approach fails to prevent undesired disulfide disruption, and a large excess of iodoacetamide causes nonspecific labeling of other residues that are sometimes visible in the UV chromatogram (Boja and Fales, 2001, Anal Chem, 73 (15): 3576-82; Muller and Winter, 2017, Mol Cell Proteomics, 16 (7): 1173-1187).

Another strategy to minimize disulfide scrambling is to conduct denaturation and digestion at acidic pH while capping free thiol with N-ethylmalcimide (NEM) due to its high reactivity in acidic conditions (Ryle et al., 1955, Biochem J, 60 (4): 541-56; Robotham and Kelly, 2019, MAbs, 11 (4): 757-766). To circumvent the low activity of trypsin in acidic pH and bolster digestion efficiency, alternative enzymes like pepsin with acceptable activities at low pH have been used, but the non-specific ragged cleavages makes the assignment of disulfide bonds rather complex. Robotham et al. further reported differentially labeling proteins with N-ethylmaleimide and d₅-N-ethylmaleimide prior to digestion.

Another solution, pioneered by Promega™ and produced as a digestion kit called AccuMAP™, utilizes rLys-C and trypsin at acidic pH to efficiently cleave arginine and lysine residues while minimizing scrambling. Since trypsin and other proteases commonly used in peptide mapping sample preparation favor alkaline pH conditions in order to efficiently digest proteins, the kit supplements trypsin with a special, low pH resistant recombinant Lys-C (rLys-C) protease. However, digestion specificity and efficiency still suffer, and a one-enzyme approach that minimizes disulfide scrambling with the high digestion specificity and efficiency of trypsin is desirable to ensure assay reproducibility and robustness.

The kinetics of iodo-based cysteine alkylations are too slow to prevent disulfide scrambling. Once a protein sample is denatured, disulfide scrambling appears inevitable in the presence of iodoacetamide/iodoacetic acid/iodoTMT. Disulfide scrambling causes the rearrangement of pre-existing free thiols into different ratios. Paired cysteines may no longer show matching free thiol levels. Maleimide-based alkylations (e.g., NEM, maleimide) can be fast enough to completely prevent disulfide scrambling from occurring, and can even label cysteines efficiently at acidic pH. The inventors discovered that a differential labeling approach can effectively maintain native free thiol levels and yield accurate, sensitive MS data for relative quantitation without the need for MS/MS fragment ion quantitation. Such a differential labeling approach employs initially labeling by alkylating a protein with a NEM analog and a second labeling by alkylating a protein digest with a heavy isotope substituted NEM analog counterpart (e.g. alkylating with maleimide and a heavy-isotope substituted maleimide, such as a carbon-13 substituted maleimide). Further, the second alkylating labeling step can be carried-out after digestion, which avoids an additional wash step to remove the alkylating agent. The disclosure herein provides such elegant workflows that can prevent disulfide bonds in proteins from scrambling during digestion conditions, e.g., tryptic digestion conditions, for example.

Three in-house IgG4 mAbs were selected and used to demonstrate how using a NEM analog and its heavy isotope substituted form in peptide mapping protocols can minimize or even eliminate disulfide scrambling artifacts. The data show that adding a NEM analog (which is less hydrophobic than maleimide for example) followed by digestive conditions and subsequently adding a heavy isotope substituted form of the NEM analog initially used in a peptide mapping protocols can essentially eliminate disulfide scrambling artifacts. These methods enable a high degree of confident analysis of proteins while maintaining the advantages of tryptic digestion, for example.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

In some exemplary implementations, the disclosure provides a method for characterizing a protein of interest. The disclosure further methods for preparing ¹³C-NEM analog alkylated products, e.g., a ¹³C-NEM analog alkylated protein of interest, a ¹³C-NEM analog alkylated peptide digest, etc. and the products per se.

As used herein, the term “protein” or “protein of interest” includes any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides' refers to a non-naturally occurring peptide or polypeptide. Synthetic peptides or polypeptides can be synthesized, for example, using an automated polypeptide synthesizer. Various solid phase peptide synthesis methods are known. A protein may contain one or multiple polypeptides to form a single functioning biomolecule. A protein can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. In another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a review discussing biotherapeutic proteins and their production, see Ghaderi et al., “Production platforms for biotherapeutic glycoproteins. Occurrence, impact, and challenges of non-human sialylation,” (BIOTECHNOL. GENET. ENG. REV. 147-175 (2012)). In some exemplary embodiments, proteins comprise modifications, adducts, and other covalently linked moieties. Those modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as, globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein can be an antibody, a bispecific antibody, a multispecific antibody, antibody fragment, monoclonal antibody, or an Fc fusion protein.

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different exemplary embodiments, the FRs of the anti-big-ET-1 antibody (or antigen-binding portion thereof) may be identical to the human germline sequences, or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. An antibody fragment may be produced by various means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

The term “Fc fusion proteins” as used herein includes part or all of two or more proteins, one of which is an Fc portion of an immunoglobulin molecule, that are not fused in their natural state. Preparation of fusion proteins comprising certain heterologous polypeptides fused to various portions of antibody-derived polypeptides (including the Fc domain) has been described, e.g., by Ashkenazi et al., Proc. Natl. Acad. Sci. USA 88:10535, 1991; Byrn et al., Nature 344:677, 1990; and Hollenbaugh et al., “Construction of Immunoglobulin Fusion Proteins,” in Current Protocols in Immunology, Suppl. 4, pages 10.19.1-10.19.11, 1992. “Receptor Fc fusion proteins” comprise one or more of one or more extracellular domain(s) of a receptor coupled to an Fc moiety, which in some embodiments comprises a hinge region followed by a CH2 and CH3 domain of an immunoglobulin. In some embodiments, the Fc-fusion protein contains two or more distinct receptor chains that bind to a single or more than one ligand(s). For example, an Fc-fusion protein is a trap, such as for example an IL-1 trap (e.g., Rilonacept, which contains the IL-1 RacP ligand binding region fused to the IL-IR1 extracellular region fused to Fc of hIgG1; see U.S. Pat. No. 6,927,004, which is herein incorporated by reference in its entirety), or a VEGF Trap (e.g., Aflibercept, which contains the Ig domain 2 of the VEGF receptor Flt1 fused to the Ig domain 3 of the VEGF receptor Flk1 fused to Fc of hIgG1; e.g., see U.S. Pat. Nos. 7,087,411 and 7,279,159, which are herein incorporated by reference in their entirety).

As used herein, the general term “post-translational modifications” or “PTMs” refers to covalent modifications that polypeptides undergo, either during (co-translational modification) or after (post-translational modification) their ribosomal synthesis. PTMs are generally introduced by specific enzymes or enzyme pathways. Many occur at the site of a specific characteristic protein sequence (signature sequence) within the protein backbone. Several hundred PTMs have been recorded, and these modifications invariably influence some aspect of a protein's structure or function (Walsh, G. “Proteins” (2014) second edition, published by Wiley and Sons, Ltd., ISBN: 9780470669853). The various post-translational modifications include, but are not limited to, cleavage, N-terminal extensions, protein degradation, acylation of the N-terminus, biotinylation (acylation of lysine residues with a biotin), amidation of the C-terminal, glycosylation, iodination, covalent attachment of prosthetic groups, acetylation (the addition of an acetyl group, usually at the N-terminus of the protein), alkylation (the addition of an alkyl group (e.g. methyl, ethyl, propyl) usually at lysine or arginine residues), methylation, adenylation, ADP-ribosylation, covalent cross links within, or between, polypeptide chains, sulfonation, prenylation, Vitamin C dependent modifications (proline and lysine hydroxylations and carboxy terminal amidation), Vitamin K dependent modification wherein Vitamin K is a cofactor in the carboxylation of glutamic acid residues resulting in the formation of a γ-carboxyglutamate (a glu residue), glutamylation (covalent linkage of glutamic acid residues), glycylation (covalent linkage glycine residues), glycosylation (addition of a glycosyl group to either asparagine, hydroxylysine, serine, or threonine, resulting in a glycoprotein), isoprenylation (addition of an isoprenoid group such as farnesol and geranylgeraniol), lipoylation (attachment of a lipoate functionality), phosphopantetheinylation (addition of a 4′-phosphopantetheinyl moiety from coenzyme A, as in fatty acid, polyketide, non-ribosomal peptide and leucine biosynthesis), phosphorylation (addition of a phosphate group, usually to serine, tyrosine, threonine or histidine), and sulfation (addition of a sulfate group, usually to a tyrosine residue). The post-translational modifications that change the chemical nature of amino acids include, but are not limited to, citrullination (the conversion of arginine to citrulline by deimination), and deamidation (the conversion of glutamine to glutamic acid or asparagine to aspartic acid). The post-translational modifications that involve structural changes include, but are not limited to, formation of disulfide bridges (covalent linkage of two cysteine amino acids) and proteolytic cleavage (cleavage of a protein at a peptide bond). Certain post-translational modifications involve the addition of other proteins or peptides, such as ISGylation (covalent linkage to the ISG15 protein (Interferon-Stimulated Gene)), SUMOylation (covalent linkage to the SUMO protein (Small Ubiquitin-related Modifier)) and ubiquitination (covalent linkage to the protein ubiquitin). See European Bioinformatics Institute Protein Information ResourceSIB Swiss Institute of Bioinformatics, EUROPEAN BIOINFORMATICS INSTITUTE DRS-DROSOMYCIN PRECURSOR-DROSOPHILA MELANOGASTER (FRUIT FLY)—DRS GENE & PROTEIN, uniprot.org/docs/ptmlist for a more detailed controlled vocabulary of PTMs curated by UniProt.

As used herein, the term “chromatography” refers to a process in which a chemical mixture carried by a liquid or gas can be separated into components as a result of differential distribution of the chemical entities as they flow around or over a stationary liquid or solid phase. Non-limiting examples of chromatography include traditional reversed-phased (RP), ion exchange (IEX), mixed mode chromatography and normal phase chromatography (NP).

As used herein, the term “mass spectrometer” includes a device capable of identifying specific molecular species and measuring their accurate masses. The term is meant to include any molecular detector into which a polypeptide or peptide may be eluted for detection and/or characterization. A mass spectrometer can include three major parts: the ion source, the mass analyzer, and the detector. The role of the ion source is to create gas phase ions. Analyte atoms, molecules, or clusters can be transferred into gas phase and ionized either separately or concurrently (as in electrospray ionization). The choice of ion source depends heavily on the application.

As used herein, the term “mass analyzer” includes a device that can separate species, that is, atoms, molecules, or clusters, according to their mass. Non-limiting examples of mass analyzers that could be employed for fast protein sequencing are time-of-flight (TOF), magnetic/electric sector, quadrupole mass filter (Q), quadrupole ion trap (QIT), orbitrap, Fourier transform ion cyclotron resonance (FTICR), and also the technique of accelerator mass spectrometry (AMS).

In some exemplary embodiments, automated iterative MS/MS can be performed under native conditions. As used herein, the term “native conditions” can include performing mass spectrometry under conditions that preserve non-covalent interactions in an analyte. For a detailed review on native MS, refer to the review: Elisabetta Boeri Erba & Carlo Pe-tosa. The emerging role of native mass spectrometry in characterizing the structure and dynamics of macromolecular complexes, Protein Science, 24(8), 1176-1192 (2015).

In some exemplary embodiments, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information or the fragment ion signal is detectable. Tandem MS have been successfully performed with a wide variety of analyzer combinations. What analyzers to combine for a certain application is determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization can include, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools.” Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

In some embodiments, the sample comprising the protein of interest in a sample can be treated by adding a reducing agent to the sample.

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, or filtration.

The method of the subject technology can be applied to any protein featuring disulfide bonds. In some exemplary embodiments, a particular application involves analysis of a protein of interest that is an antibody. In some exemplary embodiments, the protein of interest is a monoclonal antibody. In some exemplary embodiments, the protein of interest is a bispecific antibody. In some exemplary embodiments, the protein of interest is a recombinant protein.

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

A variety of denaturation agents may be used in the sample preparation step of the methods of the subject technology. Such denaturing agents include, for example, guanidine hydrochloride or urea. In some exemplary embodiments, the denaturation agent is urea. Urea can be used at a concentration of from about 5 M to about 10 M, such as from about 6 M to about 10 M. In some exemplary embodiments, urea can be used at a concentration of about 6 M, about 6.1 M, about 6.2 M, about 6.3 M, about 6.4 M, about 6.5 M, about 6.6 M, about 6.7 M, about 6.8 M, about 6.9 M, about 7 M, about 7.1 M, about 7.2 M, about 7.3 M, about 7.4 M, about 7.5 M, about 7.6 M, about 7.7 M, about 7.8 M, about 7.9 M, about 8 M, about 8.1 M, about 8.2 M, about 8.3 M, about 8.4 M, about 8.5 M, about 8.6 M, about 8.7 M, about 8.8 M, about 8.9 M, about 9 M, about 9.1 M, about 9.2 M, about 9.3 M, about 9.4 M, about 9.5 M, about 9.6 M, about 9.7 M, about 9.8 M, about 9.9 M, or about 10 M. In some exemplary embodiments, the concentration of urea is about 8 M.

Denaturation may be conducted in a variety of conditions.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, Journal of Proteome Rescarch, 12, 1067-1077 (2013)).

Method of the present disclosure advantageously can characterize a protein of interest, while minimizing or preventing the formation of sample preparation-induced disulfide scrambling. Such methods include differentially alkylating a sample with a N-ethylmaleimide (NEM) analog and its heavy isotope substituted counterpart. For example, methods of the present disclosure include alkylating a protein of interest in a first sample with an initial alkylating agent to form an alkylated protein of interest. The alkylated protein of interest is then subjected to enzyme digestion to form a peptide digest and the peptide digest is alkylated with a differential alkylating agent.

As used herein, the term “alkylation agent” refers the initial alkylating agent of either: (i) a NEM analog, or (ii) a heavy isotope substituted form of the NEM analog used to alkylate the protein of interest. A “differential alkylating agent” refers either the (i) NEM analog, or (ii) the heavy isotope substituted form of the NEM analog that was not initially used to alkylate the protein of interest. That is, if the initial alkylating agent employed to alkylate the protein of interest is (i) the NEM analog, the differential alkylating agent employed to alkylate the peptide digest is (ii) the heavy isotope substituted form of the NEM analog. Conversely, if the initial alkylating agent employed to alkylate the protein of interest is (ii) the heavy isotope substituted form of the NEM analog, the differential alkylating agent employed to alkylate the peptide digest is (i) the NEM analog. For most analysis, it is beneficial to alkylate the protein of interest with NEM analog initially and to alkylate the peptide digest with the heavy isotope substituted form of the NEM analog. This is because the abundance of free thiols on the protein of interest is typically less than the abundance of free thiols in a peptide digest, particularly after reduction. Such a sequence typically avoids overlap between the two isotopic profiles observed in mass spectra.

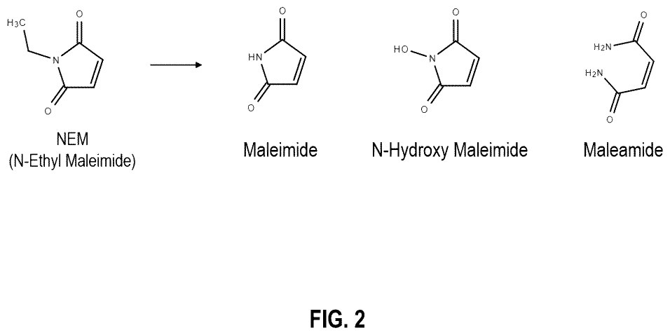

In some exemplary embodiments, the alkylation agent or differential alkylating agent is a NEM analog, such as maleimide. Maleimide can be used at a relatively wide range of concentrations such as from about 1 mM to about 20 mM. The concentration of maleimide can be about 1 mM, about 1.1 mM about 1.2 mM, about 1.3 mM, about 1.4 mM, about 1.5 mM, about 1.6 mM, about 1.7 mM, about 1.8 mM, about 1.9 mM, about 2 mM, about 2.1 mM, about 2.2 mM, about 2.3 mM, about 2.4 mM, about 2.5 mM, about 2.6 mM, about 2.7 mM, about 2.8 mM, about 2.9 mM, about 3 mM, about 3.1 mM, about 3.2 mM, about 3.3 mM, about 3.4 mM, about 3.5 mM, about 3.6 mM, about 3.7 mM, about 3.8 mM, about 3.9 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, or about 10 mM. In some exemplary embodiments, the concentration of maleimide is about 4.0 mM. Some examples of NEM analogs are shown in FIG. 2.

In some exemplary embodiments, a heavy isotope substituted form of the NEM analog includes a NEM analog substituted with a heavy isotope of hydrogen, carbon, nitrogen, oxygen, or any combination thereof. For example, a heavy isotope substituted form of the NEM analog can include a heavy isotope of carbon, such as carbon-13, or carbon-14; a heavy isotope of hydrogen, such as deuterium, or tritium; a heavy isotope of nitrogen such as nitrogen-15; a heavy isotope of oxygen such as oxygen-17, or oxygen-18; or any combination thereof. The heavy isotope substituted form of the NEM analog includes heavy isotopes in an amount that is in excess of naturally occurring isotopes, e.g., in an amount in excess of about 1.5%, such as in an amount in excess of 25% or 50% or higher. While a heavy isotope substituted form of NEM analog can include a variety of one or more heavy isotopes, it was found that near identical chromatographic retention times can be achieve when using a heavy carbon substituted form of the NEM analog relative the NEM analog. As shown by the examples below, a deuterated substituted form of the NEM analog had a shifted chromatographic retention time relative to its non-deuterated form. Hence, in certain implementations of the present disclosure, the heavy isotope substituted form of the NEM analog includes a carbon-13 and/or carbon-14 substituted NEM analog such as a carbon-13 and/or carbon-14 substituted form of maleimide, e.g., a fully carbon-13 substitute maleimide (i.e., ¹³C₄-labelled maleimide).

Advantageously, each of alkylating the protein of interest, or alkylating the peptide digest with either (i) a NEM analog, or (ii) a heavy isotope substituted form of the NEM analog can be carried out under acidic conditions, e.g., pH<7, such as from a pH of about 3 to a pH of about 6.5. Such acidic conditions can minimize disulfide scrambling and minimize over alkylation, e.g., alkylating amino groups of the proteins or peptides in addition to thiol groups.

Moreover, the second alkylation (alkylating the peptide digest) can be carried out after digesting the alkylated protein of interest. For example, digestion can be carried out at any pH suitable for the digestive enzyme since it can be a separate step after the initial alkylation including digesting alkaline conditions. The digestion can be quenched with an acid followed by alkylating the peptide digest (the second alkylation step). As such the quenched digest can have an acidic medium. And by performing the second alkylation under acidic conditions after digestion has been quenched, there is minimal risk of over-alkylation. Such a workflow further minimizes the number of wash steps.

Digestive enzymes that are useful for the methods of the subject technology, such as for peptide mapping, can include, for example, one or more of trypsin, pepsin, or LysC. In some exemplary embodiments, the digestive enzyme comprises or consists of trypsin. Trypsin can be used at an enzyme: substrate ratio of about 1:5, about 1:5.5, about 1:6, about 1:6.5, about 1:7, about 1:7.5, about 1:8, about 1:8.5, about 1:9, about 1:9.5, about 1:10, about 1:10.5, about 1:11, about 1:11.5, about 1:12, about 1:12.5, about 1:13, about 1:13.5, about 1:14, about 1:14.5, about 1:15, about 1:16, about 1:17, about 1:18, about 1:19, or about 1:20. In some exemplary embodiments, the enzyme: substrate ratio of trypsin is about 1:10. In some aspects, digestion does not induce reduction of disulfide bonds.

Digestion can be conducted in a pH that can range from acidic (e.g., <pH 7) to alkaline (e.g., >pH 7), such as pH from about 3 to about 9. For example, digestion can be conducted at a pH of about 5, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In some exemplary embodiments, the pH for digestion is about 7.5.

While the methods described above recite certain characterizations of at least one disulfide bond of a protein of interest, it should be understood that the methods can be extended to a variety of applications. It is further understood that “characterizing” at least one protein of interest can include, for example, identifying, quantifying, and/or comparing said at least one protein of interest.

It is understood that the present invention is not limited to any of the aforesaid protein(s), protein(s) of interest, antibody(s), protein alkylating agent(s), protein denaturing agent(s), protein reducing agent(s), digestive enzyme(s), sample(s), chromatographic method(s), mass spectrometer(s), database(s), bioinformatics tool(s), pH, temperature(s), or concentration(s), and any protein(s), protein(s) of interest, antibody(s), protein alkylating agent(s), protein denaturing agent(s), protein reducing agent(s), digestive enzyme(s), sample(s), chromatographic method(s), mass spectrometer(s), database(s), bioinformatics tool(s), pH, temperature(s), or concentration(s). Such features or parameters can be selected and adjusted to suit the particular method practiced given the guidance of the present disclosure and understanding in the art.

The consecutive labeling of method steps as provided herein with numbers and/or letters is not meant to limit the method or any embodiments thereof to the particular indicated order.

Various publications, including patents, patent applications, published patent applications, accession numbers, technical articles and scholarly articles are cited throughout the specification.

The disclosure will be more fully understood by reference to the following Aspects and Examples, which are provided to describe the disclosure in greater detail. They are intended to illustrate examples and should not be construed as limiting the scope of the disclosure.

### Aspects

Aspect 1. A method for site-specific free thiol quantitation in a sample including a protein of interest, said method comprising:
a. contacting said sample to a NEM analog to form alkylated protein of interest;
b. contacting said alkylated protein of interest to at least one digestive enzyme to form a peptide digest;
c. contacting said peptide digest to a heavy isotope labeled form of the NEM analog to form a second sample; and
d. subjecting said second sample to analysis using liquid chromatography-mass spectrometry to quantify the site-specific free thiol in the first sample.

Aspect 2. The method of Aspect 1, further comprising performing a buffer exchange after step (a).

Aspect 3. The method of Aspect 1, further comprising adding a reducing agent to the peptide digest.

Aspect 4. The method of Aspect 3, wherein the reducing agent is Tris(2-carboxyethyl)phosphine.

Aspect 5. The method of Aspect 1, wherein the NEM analog is less hydrophobic than NEM.

Aspect 6. The method of Aspect 1, wherein the NEM analog has a retention time less than retention time of NEM.

Aspect 7. The method of Aspect 1, wherein the NEM analog is maleimide.

Aspect 8. The method of Aspect 1, wherein the concentration of NEM analog used to contact said sample is about 1 mM to about 10 mM.

Aspect 9. The method of Aspect 1, wherein the concentration of NEM analog used to contact said sample is about 2 mM to about 8 mM.

Aspect 10. The method of Aspect 1, wherein the concentration of NEM analog used to contact said sample is about 4 mM.

Aspect 11. The method of Aspect 1, wherein said at least one digestive enzyme is trypsin.

Aspect 12. The method of Aspect 1, wherein said at least one digestive enzyme is Lys-C.

Aspect 13. The method of Aspect 1, wherein said at least one digestive enzyme is Lys-C and trypsin.

Aspect 14. The method of Aspect 1, wherein said digestion is conducted at a pH between about 7 and about 8.

Aspect 15. The method of Aspect 1, wherein said digestion is conducted at a pH between about 7 and about 7.5.

Aspect 16. The method of Aspect 1, wherein said digestion is conducted at a pH between about 5 and about 6.

Aspect 17. The method of Aspect 1, wherein said digestion is conducted at a pH between about 5.3 and about 7.

Aspect 18. A method for characterizing a protein of interest in a first sample, said method comprising:
a. contacting said first sample to a NEM analog to form a second sample with an alkylated protein of interest;
b. contacting said alkylated protein of interest to at least one digestive enzyme to form a peptide digest; and
c. contacting said peptide digest to a heavy isotope labeled form of the NEM analog to form a third sample; and
d. subjecting said third sample to analysis using liquid chromatography-mass spectrometry to characterize said protein of interest in the first sample.

Aspect 19. A method for performing a non-reduced peptide mapping of a protein of interest in a first sample, said method comprising:
a. contacting said first sample to a NEM analog to form a second sample with an alkylated protein of interest;
b. contacting said alkylated protein of interest to at least one digestive enzyme to form a peptide digest; and
c. contacting said peptide digest to a heavy isotope labeled form of the NEM analog to form a third sample; and
d. subjecting said third sample to analysis using liquid chromatography-mass spectrometry to obtain said non-reduced peptide mapping of said protein of interest.

### EXAMPLES

### Materials

Urea was purchased from Sigma-Aldrich (St. Louis, MO). Tris-HCl buffer, pH 7.5 was obtained from Invitrogen (Carlsbad, CA). Purified monoclonal antibodies were produced internally by Regeneron (Tarrytown, NY). Maleimide and ¹³C₄-maelimide was obtained from Sigma-Aldrich.

Regular Peptide Mapping Method with IAM and NEM.

For peptide mapping sample preparation, an aliquot of mAb1 sample was denatured and alkylated with IAM and/or NEM. This solution was centrifuged at 14,000 rpm for 10 min, washed three times with 100 μL of water (14,000 rpm for 10 min each time). To this, 20 μL of 8 M urea in 100 mM Tris-HCl (pH 7.0) was added and incubated at 50° C. for 10 min. This sample was diluted with 75 μL of 100 mM Tris-HCl (pH 7.0) and 5 μL of 1 mg/mL trypsin was added, and incubate at 42° C. for 3 h. Finally, 2 μL of 10% TFA was added to quench the reaction followed by 5 μL of 100 mM TCEP, and incubated at 50° C. for 20 min.

Modified Peptide Mapping Method with Using Heavy Isotope Labelled Maleimide.

For peptide mapping sample preparation with the heavy isotope labelled NEM analog, maleimide and ¹³C₄ maleimide were employed. Initially a 300 μg original protein sample was dilute with 100 μL of 5 mM acetic acid+10 mM maleimide, and incubated 80° C. for 15 min. This step denatures the protein and labels pre-existing free thiols in the protein sample. Using 10K Nanosep, the sample was centrifuged at 14,000 rpm for 10 min and then washed three times with 100 μL of water (14,000 rpm for 10 min each time) until the filter was entirely dry. The buffer-exchange step removes excess maleimide present in the sample. The sample is then reconstituted with 60 μL of water and its concentration is measured in NanoDrop.

100 μg of this maleimide-labeled protein is transferred to an Eppendorf tube. Lyophilize in SpeedVac (˜15 min) wot which 20 μL of 8 M urea in 100 mM Tris-HCl (pH 7.0) is added, and incubated at 50° C. for 10 min. This would denature the protein again, in case of natural refolding. The sample is then diluted with 75 μL of 100 mM Tris-HCl (pH 7.0) to which 5 μL of 1 mg/mL trypsin (+2 μL of 1 mg/mL LysC, if mAb is IgG1) is added, and incubated at 42° C. for 3 h to form a non-reduced peptide mapping digest. To the non-reduced peptide mapping digest, 1 μL of 10% TFA, followed by add 5 μL of 100 mM TCEP is added, and incubated at 50° C. for 20 min. This lowers the pH to less than 3, quenches digestive enzyme, and reduces disulfide peptides.

Finally, 5 μL of 300 mM ¹³C₄-maleimide is added to the sample, and incubated at 50° C. for 20 min. The ¹³C₄-maleimide labels TCEP-reduced cysteines. 2 μL of 10% TFA is added after to quench the digestion and/or labeling. 6 μL (˜5 μg of protein) is then injected into LC/MS for site-specific free thiol quantitation.

### LC/UV-MS Analysis.

A Waters ACQUITY UPLC I-Class system coupled to a Thermo Scientific Q Exactive Plus mass spectrometer was used to analyze the non-reduced digested samples. The tryptic peptide mixture was separated by a Waters ACQUITY UPLC BEH® 130 C18 column (1.7 μm, 2.1 mm×150 mm) at a flow rate of 0.25 mL/minute. Mobile phase A was 0.05% TFA in water and mobile phase B was 0.045% TFA in acetonitrile. The gradient was held at 0.1% B for the first 5 minutes and then increased to 26% B in 55 minutes followed by another increase to 34.5% B in 35 minutes. The column was equilibrated with 99.9% mobile phase A prior to sample injection, with the column temperature maintained at 40° C. The MS data were acquired on a Thermo Scientific Q Exactive Plus mass spectrometer from m/z 300-2000 at a resolution of 70 k (at m/z 400), followed by five data-dependent MS/MS scans at a resolution of 17.5 k. MS full scans were set at 1×10⁶ automated gain control (AGC) and a maximum injection time of 50 ms. MS² fragmentation was performed using HCD with a normalized collision energy of 28% at a 1×10⁵ AGC, and a maximum injection time of 100 ms. Dynamic exclusion duration was set to 15 seconds with a single repeat count.

### Data Analysis

All peptide identity assignments and post-translational modification identifications were performed using Protein Metrics Byonic™ (version 3.11.3) by searching the raw files against the mAb protein sequence. The preliminary list of unique peptides was generated by filtering against a 1% FDR. The list of precursors and the original searching results as a spectra library were then imported into Skyline Daily software (University of Washington, WA) for a full scan (MS¹)-based final ID validation. The peak area was extracted by summing all charge states through Skyline software.

### Example 1. Kinetic Study in Alkylation Methods Using IAM and NEM

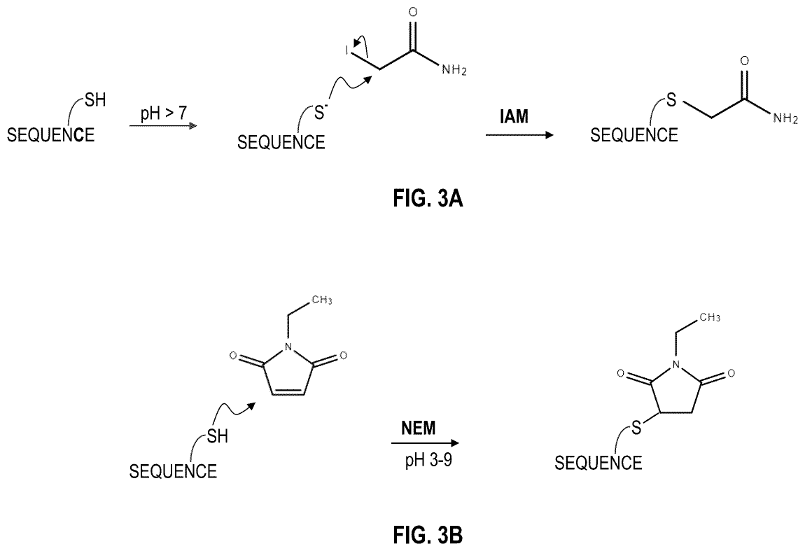

Protein characterization of mAb1 (IgG1 antibody) was carried out using the peptide mapping with both, IAM and NEM. IAM and NEM have distinct alkylation mechanism for alkylating free thiols. See FIGS. 3A and 3B.

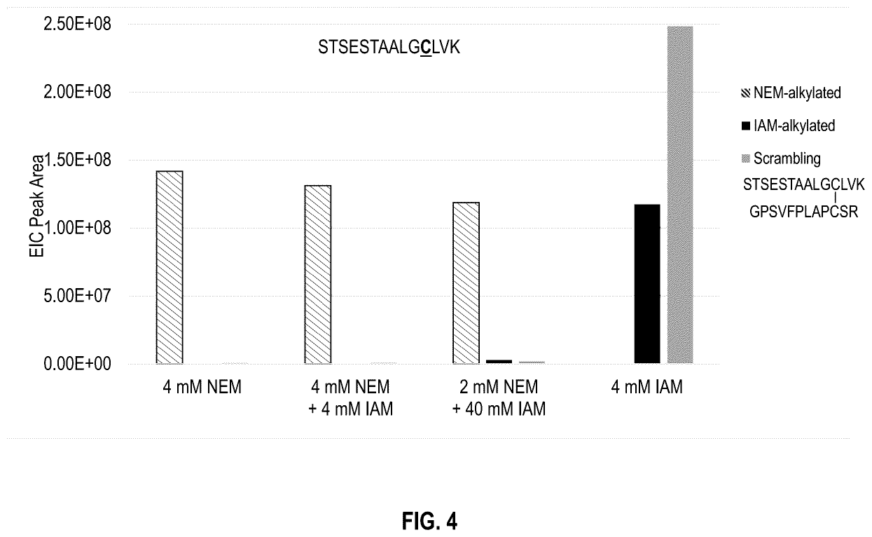

Disulfide scrambling was evaluated for methods using 4 mM NEM alone, 4 mM MEM+4 mM IAM, 2 mM NEM+40 mM IAM, and 4 mM IAM alone. It was found that use of NEM alone or when added to IAM minimized disulfide scrambling. See FIG. 4. The use of IAM, instead, induced significant disulfide scrambling. Further, even when an enormous excess of IAM (40 mM; third column in the chart in FIG. 4) is spiked in with NEM, the vast majority of alkylated peptide is in the NEM-form. This shows that the reaction kinetics of NEM (or its other analogs, such as, maleimides) are significantly faster than IAM, and therefore capable of alkylating free thiols before they can induce disulfide scrambling.

### Example 2. Labelled NEM and Maleimide Alkylation for Non-Reducing Peptide Mapping Method

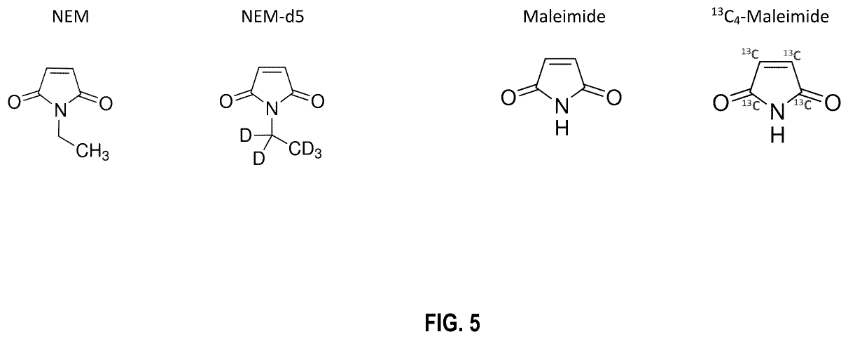

NEM is used widely in the industry as an alkylating agent. It performs fast and complete alkylation due to high reactivity of its strained ring structure. See FIG. 5 for chemical structures of NEM, maleimide, and their exemplary heavy isotope labeled counterparts. Further, any protocol-induced disulfide scrambling is almost completely blocked at acidic pH levels. The NEM dominant peak with a retention time of 20 minutes and a strong UV signal however, poses a concern since it interferes with the chromatogram, as seen for mAb1.

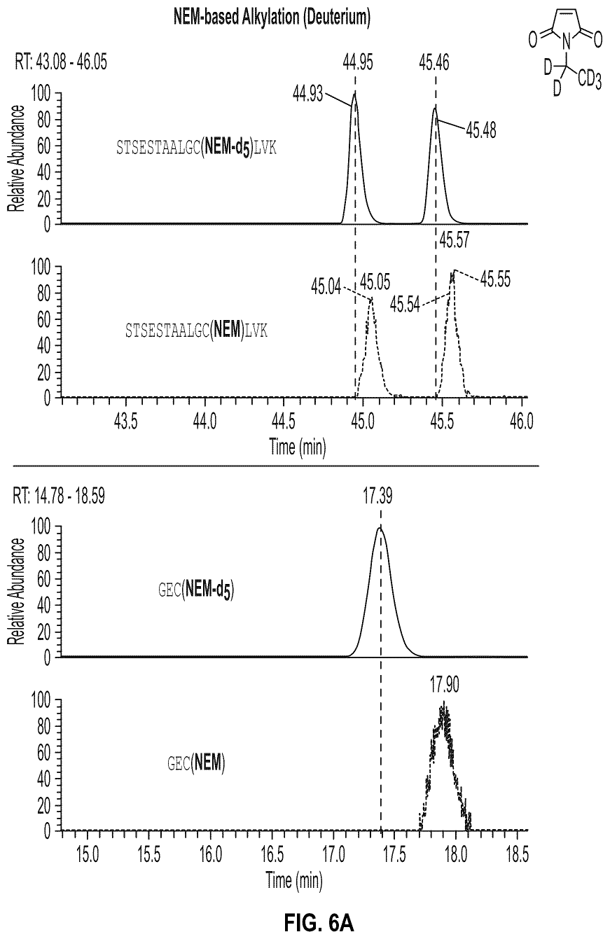

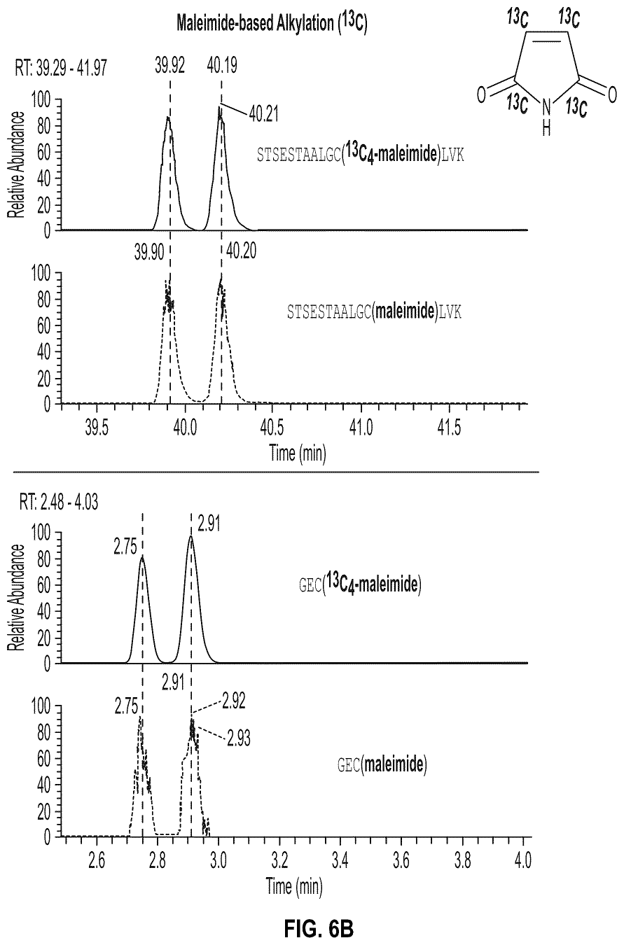

The 20 minute retention time can be theoretically reduced by reducing the hydrophobicity of NEM while maintaining its high reactivity and stability as an alkylating agent by using maleimide. FIG. 6 shows the reduction in retention time of maleimide versus NEM. The modified method for non-reduced peptide mapping requires the use of labelling and when labeled NEM and labeled maleimide were compared, it was evident that labelled-maleimide was more favorable to use. See FIG. 6B. ¹³C-labeling in maleimide introduces essentially no difference in hydrophilicity compared to maleimide, providing near perfect retention time alignment (FIG. 6B). Contrarily, d₅-labeling of NEM significantly shifts the chromatographic retention time when compared to NEM (FIG. 6A).

### Example 3. Evaluating the Purity of ¹³C₄-Maleimide

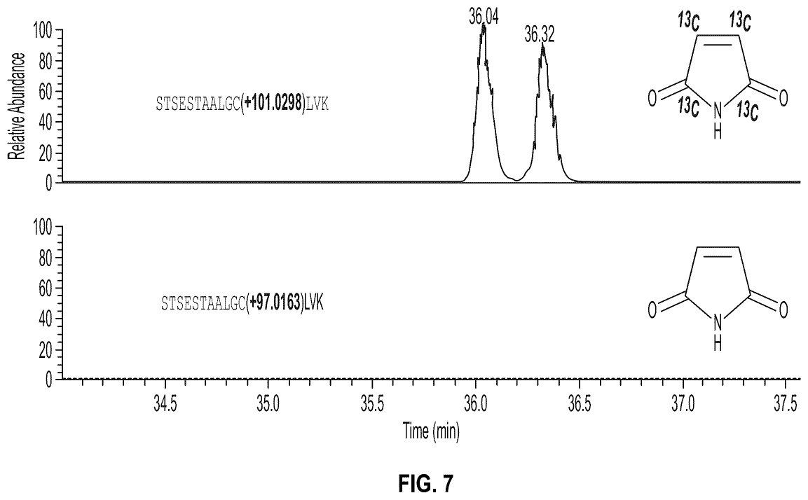

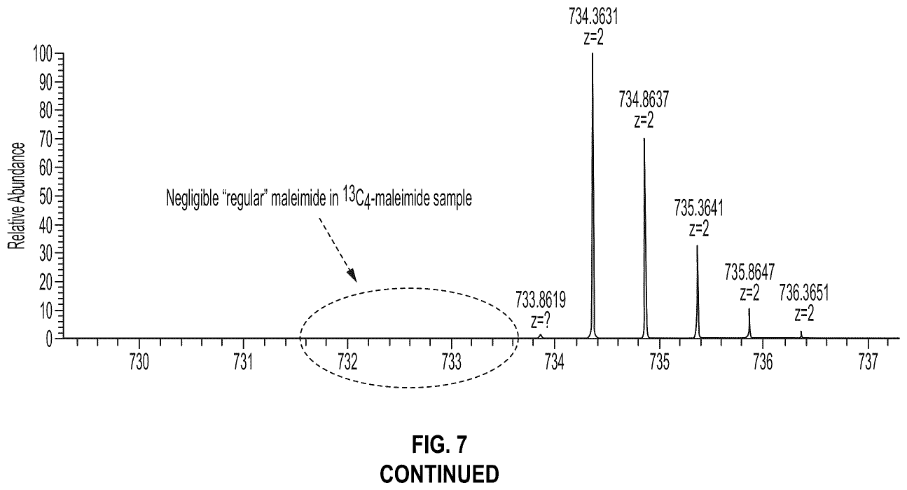

Custom synthesized ¹³C₄-substituted maleimide (from Sigma-Aldrich) was found to be nearly 100% isotopically pure when comparing mono-isotopic m/z with unlabeled maleimide (FIG. 7). That is, the synthesized ¹³C₄-substituted maleimide appears to contain a small amount of ¹³C₃-maleimide, negligible amounts of ¹³C₂ and ¹³C₁-maleimide, and none of ¹³C₀ (unsubstituted) maleimide. The second and third isotopic peaks were found to be >99.9% pure and the fourth isotopic peak was found to be about 99% pure. For Free thiol % calculations, integrations can only include up to the third isotopic m/z peak for accurate results, but this data is enough to provide accurate results.

### Example 4. Free Thiol Calculations

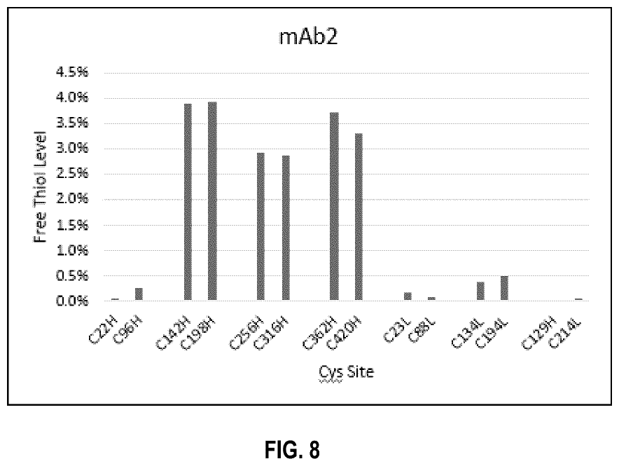

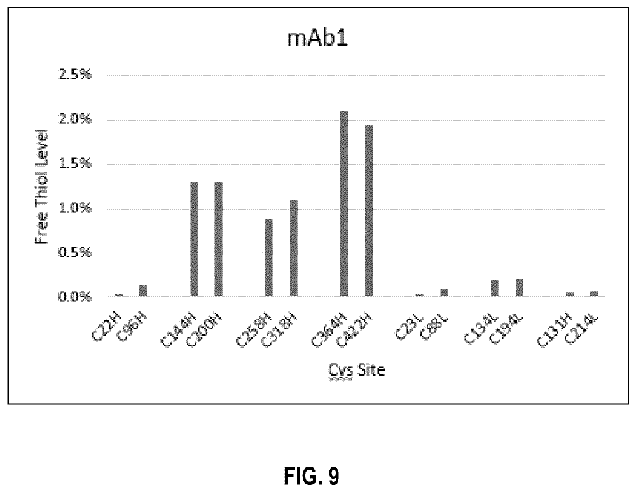

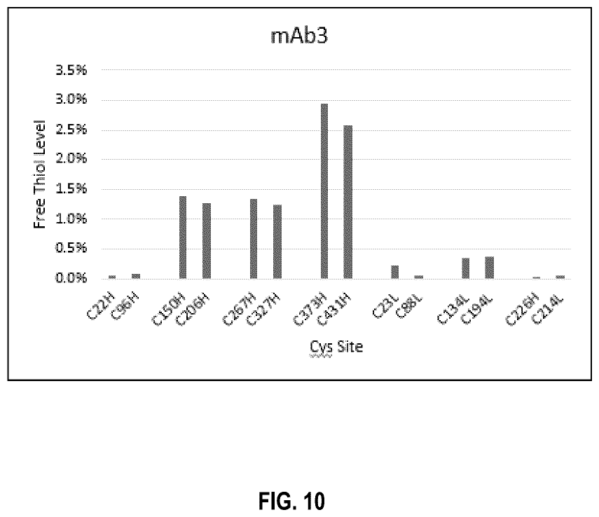

Protein characterization of mAb2 (IgG4 antibody), mAb1 (IgG4 antibody), and mAb3 (IgG1 antibody) was carried out as per the modified peptide mapping method with using heavy isotope labelled maleimide. As shown in FIGS. 8-10, the free thiol levels show that all the cysteine paired well suggesting no scrambling.

The methods disclosed perform the non-reduced mapping to quantify site-specific free thiol in a protein of interest take about 6 hours of preparation time, which is significantly lower than other traditional methods. Further, the methods do not require any additional independent data analysis.

Only certain features and aspects of the subject technology and examples of its versatility are shown and described in the present disclosure. It is to be understood that the technology disclosed herein is capable of use in various other combinations and environments and is capable of changes or modifications. Thus, for example, those skilled in the art will recognize, or be able to ascertain, using no more than routine experimentation, numerous equivalents to the specific substances, procedures and arrangements described herein. Such equivalents are considered to be within the scope of the invention, and are covered by the following claims.

Sequence Table

| SEQ ID NO | Sequence |
|---|---|
| 1 | STSESTAALGCLVK |
| 2 | GPSVFPLAPCSR |
| 3 | STSESTAALGC(NEM-d₅)LVK |
| 4 | STSESTAALGC(NEM)LVK |
| 5 | STSESTAALGC(¹³C₄-maleimide)LVK |
| 6 | STSESTAALGC (maleimide)LVK |

What is claimed is:

1. A method for site-specific free thiol quantitation in a first sample including a protein of interest, the method comprising:
alkylating the protein of interest in the first sample with an alkylating agent to form a second sample with an alkylated protein of interest, wherein the alkylating agent is either: (i) a NEM analog, or (ii) a heavy isotope substituted form of the NEM analog;
digesting the alkylated protein of interest with at least one digestive enzyme to form a peptide digest;
alkylating the peptide digest with a differential alkylating agent to form a third sample, wherein the differential alkylating agent is either (i) the NEM analog, or (ii) the heavy isotope substituted form of the NEM analog; and
subjecting the third sample to analysis using liquid chromatography-mass spectrometry to quantify the site-specific free thiol in the first sample.

2. A method for characterizing a protein of interest in a first sample, the method comprising:
alkylating the protein of interest in the first sample with an alkylating agent to form a second sample with an alkylated protein of interest, wherein the alkylating agent is either: (i) a NEM analog, or (ii) a heavy isotope substituted form of the NEM analog;
digesting the alkylated protein of interest with at least one digestive enzyme to form a peptide digest;
alkylating the peptide digest with a differential alkylating agent to form a third sample, wherein the differential alkylating agent is either: (i) the NEM analog, or (ii) the heavy isotope substituted form of the NEM analog; and
subjecting the third sample to analysis using liquid chromatography-mass spectrometry to characterize the protein of interest in the first sample.

3. The method of claim 2, wherein characterizing the protein of interest comprises subjecting the third sample to analysis using liquid chromatography-mass spectrometry to obtain a non-reduced peptide mapping of the protein of interest.

4. The method of claim 1, further comprising performing a buffer exchange after alkylating the protein of interest in the first sample.

5. The method of claim 1, further comprising, prior to alkylating the peptide digest with the differential alkylating agent, reducing the peptide digest with a reducing agent.

6. The method of claim 5, wherein the reducing agent comprises tris(2-carboxyethyl)phosphine.

7. The method of claim 1, wherein the NEM analog is less hydrophobic than NEM.

8. The method of claim 1, wherein the alkylating agent is the heavy isotope substituted form of the NEM analog and the differential alkylating agent is the NEM analog.

9. The method of claim 1, wherein the alkylating agent is the NEM analog and the differential alkylating agent is the heavy isotope substituted form of the NEM analog.

10. The method of claim 1, wherein the at least one digestive enzyme comprises trypsin, Lys-C, or a combination thereof.

11. The method of claim 1, wherein the at least one digestive enzyme consists of trypsin.

12. The method of claim 1, wherein the heavy isotope substituted form of the NEM analog comprises a heavy isotope of carbon substituted form of the NEM analog.

13. The method of claim 1, wherein the heavy isotope substituted form of the NEM analog comprises a carbon 13 substituted form of the NEM analog.

14. The method of claim 1, wherein digesting the alkylated protein of interest is conducted at a pH between about 7 and about 8.

15. The method of claim 1, wherein alkylating the protein of interest in the first sample with an alkylating agent, and alkylating the peptide digest with a differential alkylating agent are each conducted under acidic conditions.

16. The method of claim 1, wherein the protein of interest is an antibody.

17. A method for characterizing a protein of interest in a first sample, the method comprising:
forming an alkylated product by either: alkylating the protein of interest with a carbon 13 substituted NEM analog to form a ¹³C-NEM analog alkylated protein of interest, or alkylating a peptide digest of the protein of interest with a carbon 13 substituted NEM analog to form a ¹³C-NEM analog alkylated peptide digest.

18. The method of claim 17, wherein the carbon 13 substituted NEM analog comprises ¹³C₄-maleimide.

19. The method of claim 17, further comprising: subjecting the alkylated product to analysis using liquid chromatography-mass spectrometry to characterize the protein of interest.

20. The alkylated product obtained by the method of claim 17.


SEQUENCE LISTING (6 sequences)

SEQ ID NO: 1 - protein, 14 aa, synthetic construct
STSESTAALGCLVK

SEQ ID NO: 2 - protein, 12 aa, synthetic construct
GPSVFPLAPCSR

SEQ ID NO: 3 - protein, 14 aa, synthetic construct
  SITE 11: note = N-Ethyl-d5-maleimide modified residue
STSESTAALGCLVK

SEQ ID NO: 4 - protein, 14 aa, synthetic construct
  SITE 11: note = N-Ethylmaleimide modified residue
STSESTAALGCLVK

SEQ ID NO: 5 - protein, 14 aa, synthetic construct
  SITE 11: note = 13C4-labeled maleimide modified residue
STSESTAALGCLVK

SEQ ID NO: 6 - protein, 14 aa, synthetic construct
  SITE 11: note = Maleimide modified residue
STSESTAALGCLVK